(19) United States
(12) Patent Application Publication
(10) Pub. No.: US 2021/0301438 A1
(43) Pub. Date: Sep. 30, 2021

(54) SPUNBOND WIPE AND MANUFACTURING METHOD THEREOF

(71) Applicant: XIAMEN YANJAN NEW MATERIAL CO., LTD., Xiamen, Fujian (CN)

(72) Inventors: Qingzhong Zheng, Xiamen (CN); Shunxin He, Xiamen (CN); Danpin Wu, Xiamen (CN); Jihua Xie, Xiamen (CN)

(21) Appl. No.: 17/262,210

(22) PCT Filed: May 17, 2019
(86) PCT No.: PCT/CN2019/087397

§ 371 Date: Jan. 22, 2021

(30) Foreign Application Priority Data
Jan. 15, 2019 (CN) 201910036664.6

(51) Int. Cl. D04H 1/56; D04H 1/26; D01D 5/098; A61F 13/15

(52) U.S. Cl. CPC D04H 1/56; D04H 1/26; A61F 2013/16; A61F 13/15; D01D 5/098

(57) ABSTRACT

A spunbond wipe is provided. At least one of upper and lower layers of the spunbond wipe is a spunbond long fiber layer. An intermediate layer of the spunbond wipe is a wood pulp fiber web. Spunbond long fibers of the spunbond long fiber layer are interwoven in the wood pulp fiber web. Also provided is a manufacturing method of the spunbond wipe.

### BACKGROUND OF THE INVENTION

### 1. Field of the Invention

The present invention relates to a wipe, and more particularly to a spunbond wipe with soft and wear-resistant surfaces for personal hygiene and baby care. A manufacturing method for the wipe is also disclosed.

### 2. Description of the Prior Art

Wipes are often used in our daily life when, for example, parents change babies' diapers, people have meals in restaurants, or people do skincare. Since wipes are convenient to carry, store and use, they are popular among consumers and having increasingly extensive use in particularly personal hygiene and baby care.

Wipes can be used in a dry or wet manner to wipe off dust and stains, and can work with water or detergent to remove stubborn stains. For improving wipes in terms of moisture absorption and water absorption, a common solution is to add wood pulp during production of wipe products in order to make wipes more effective in moisture absorption and cleaning. However, wood pulp fibers are natural short fibers, which are prone to shed fibers due to friction during use, and thus remain on the surface of the object to be wiped, resulting in poor wiping effect.

### SUMMARY OF THE INVENTION

The objective of the present invention is to address shortcomings of the existing products and manufacturing methods by providing a spunbond wipe with soft and wear-resistant surfaces, and providing a manufacturing method therefor.

For achieving the foregoing objective, the present invention implements the following technical schemes.

A spunbond wipe comprises upper and lower layers and an intermediate layer of a wood pulp fiber web. At least one of the upper and lower layers is a spunbond long fiber layer. Spunbond long fibers of the spunbond long fiber layer are woven into the wood pulp fiber web.

One of the upper and lower layers is the spunbond long fiber layer, and the other is a melt-blown fiber layer.

The spunbond long fiber layer contains one-component spunbond fibers each having a high-melting-point resin at its surface, spunbond fibers each having a low-melting-point resin at its surface, or blend fibers of the two.

The high-melting-point resin and the low-melting-point resin have a melting-point difference therebetween of ≥20° C.

The spunbond fibers each having the low-melting-point resin at its surface are one-component spunbond fibers, two-component spunbond fibers, or a blend of the two.

The two-component spunbond fibers are two-component skin-core spunbond fibers, two-component segmented-pie spunbond fibers, or two-component side-by-side spunbond fibers.

The melt-blown fiber layer contains one-component melt-blown fibers each having a high-melting-point resin at its surface, fibers each having a low-melting-point resin at its surface, or blend fibers of the two.

The fibers each having the low-melting-point resin at its surface are one-component melt-blown fibers, two-component melt-blown fibers, or a blend of the two.

The two-component melt-blown fibers are two-component skin-core melt-blown fibers, two-component segmented-pie melt-blown fibers, or two-component side-by-side melt-blown fibers.

A weight percentage of the wood pulp fiber web in the spunbond wipe is greater than 50 wt %.

A weight percentage of the wood pulp fiber web in the spunbond wipe is 65 wt %-80 wt %.

The wood pulp fiber web contains a hot-melt adhesive substance. The hot-melt adhesive substance is polyester fibers having a low-melting-point, ES short fibers, two-component spunbond long fibers or a blend thereof.

The wood pulp fiber web contains a super absorbent resin.

An adhesive is attached to surfaces of the upper and lower layers of the spunbond wipe.

An adhesive is attached to surfaces of the upper and lower layers of the spunbond wipe and to interiors of the upper and lower layers that are adjacent to the surfaces.

The adhesive is hot-melt adhesive or solvent adhesive.

A manufacturing method for the foregoing spunbond wipe comprises steps of:

(1) opening and scattering wood pulp using an opening roller, and forming a wood pulp fiber web using a blowing nozzle in the presence of an auxiliary air stream;

(2) performing a spunbond process for one side of the wood pulp fiber web, involving heating and melting a thermoplastic resin to enter a spinning device; in the spinning device, dispersing the high-temperature thermoplastic resin into melt streams to be extruded from spinneret plates and cooled by side blowing of cold air; stretching spunbond fibers using stretching devices; and joining the spunbond fibers with two side surfaces of the wood pulp fiber web, so as to form a multi-layer fiber web having the spunbond long fiber layer at its at least one side and having the wood pulp fiber web in its middle; and

(3) binding the fiber webs in the multi-layer fiber web together using a heating device, so as to form the spunbond wipe having one least one of the upper and lower layers of the spunbond long fiber layer and having the intermediate layer of the wood pulp fiber web.

In the step (2), the spunbond process is performed for one side of the wood pulp fiber web, and a melt blowing process is performed for another side of the wood pulp fiber web.

In the step (2), the spunbond process is performed for one side of the wood pulp fiber web, involving heating and melting two thermoplastic resins having a melting-point difference therebetween of ≥20° C. to enter the spinning device; in the spinning device, dispersing the high-temperature thermoplastic resins into melt streams to be extruded from the spinneret plates; cooling by side blowing of the cold air to form the spunbond long fiber layer using stretching devices, wherein the spunbond long fiber layer contains fibers each having a high-melting-point resin at its surface, fibers each having a low-melting-point resin at its surface, or blend fibers of the two.

The spinneret plates have two-component spinneret orifices.

The two-component spinneret orifices of the spinneret plates are arranged in a skin-core form, a segmented-pie form, or a side-by-side form.

In the step (2), the melt blowing process is performed for the other side of the wood pulp fiber web, involving heating and melting two thermoplastic resins having a melting-point difference therebetween of ≥20° C.; dispersing melt streams of the thermoplastic resins that have been extruded from spinneret plates into fiber bundles having a diameter of ≤10 μm using a high-temperature, high-speed hot air stream, thereby forming a melt-blown fiber layer in the presence of the hot air stream, wherein the melt-blown fiber layer contains fibers each having a high-melting-point resin at its surface, fibers each having a low-melting-point resin at its surface, or blend fibers of the two.

The wood pulp that has been opened and scattered by the opening roller is mixed with a hot-melt adhesive substance into a mixture, and the mixture is blown using the blowing nozzle in the presence of the auxiliary air stream to form the wood pulp fiber web containing the hot-melt adhesive substance.

Before the step (3), an adhesive spraying process is performed using blowing nozzles in the presence of a high-pressure hot air flow to spray an adhesive on two side surfaces of the multi-layer fiber web.

After the step (3), a roll coating process is performed to coat an adhesive on two side surfaces of the multi-layer fiber web.

In the step (3), the heating device is a hot air oven, a hot rolling roller or a combination thereof.

In the step (1), the wood pulp that has been opened and scattered by the opening roller is mixed with a super absorbent resin into a mixture, and the mixture is blown using the blowing nozzle in the presence of the auxiliary air stream to form the wood pulp fiber web containing the super absorbent resin.

By adopting the aforementioned technical schemes, the present invention provides a spunbond wipe made using the manufacturing method. Since at least one surface is a spunbond long fiber layer composed of spunbond long fibers, the middle layer is a wood pulp fiber web, and the spunbond long fibers of the spunbond long fiber layer are woven into the wood pulp fiber web, so that the wood pulp fibers adjacent to the spunbond long fiber layer are retained in the spunbond long fiber layer. In addition, the spunbond long fiber layer on at least one surface has thinner fibers arranged densely, which effectively prevents wood pulp fibers from moving from the middle layer to a surface of the wipe and remaining on the object to be wiped. Besides, the spunbond long fibers are longer and finer fibers, the fibers of the surface layers are less broken and arranged densely, so that the spunbond wipe is soft and unlikely to pill and fuzz.

### BRIEF DESCRIPTION OF THE DRAWINGS

FIG. 1 is a schematic drawing illustrating manufacturing of a spunbond wipe according to Embodiment 1 of the present invention;

FIG. 2 is a cross-sectional view of the spunbond wipe according to Embodiment 1 of the present invention;

FIG. 3 is a schematic drawing illustrating manufacturing of a spunbond wipe according to Embodiment 2 of the present invention;

FIG. 4 is a cross-sectional view of the spunbond wipe according to Embodiment 2 of the present invention;

FIG. 5A is a cut-away view of a skin-core melt-blown fiber according to the present invention;

FIG. 5B is a cut-away view of a two-component side-by-side melt-blown fiber according to the present invention;

FIG. 5C is a cut-away view of a two-component segmented-pie melt-blown fiber according to the present invention;

FIG. 6 is a cross-sectional view of the spunbond wipe according to Embodiment 3 of the present invention;

FIG. 7 is a cross-sectional view of the spunbond wipe according to Embodiment 4 of the present invention; and

FIG. 8 is a cross-sectional view of the spunbond wipe according to Embodiment 5 of the present invention.

### DETAILED DESCRIPTION OF THE PREFERRED EMBODIMENTS

For further illustrating the technical schemes of the present invention, the following detailed description of the present invention will be made with reference to particular embodiments

### Embodiment 1

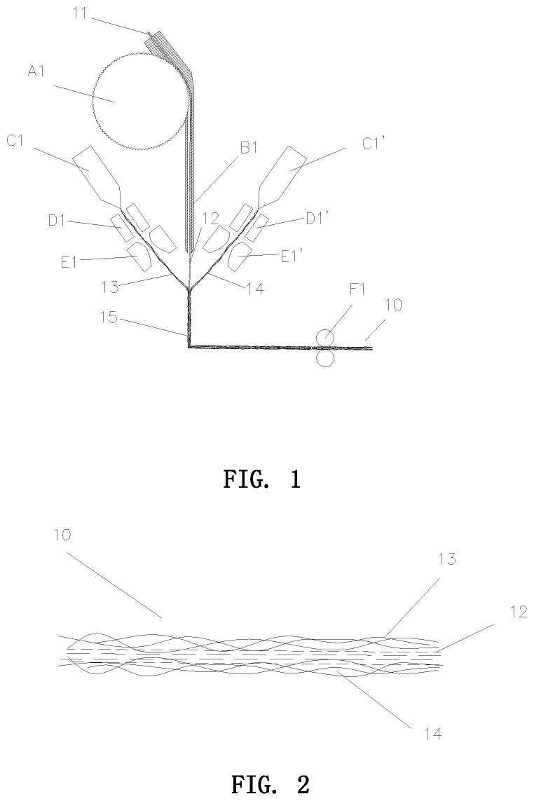

As shown in FIG. 1, the present invention discloses a manufacturing method for a spunbond wipe, comprising the following steps of:

(1) opening and scattering wood pulp 11 using an opening roller A1, and forming a wood pulp fiber web 12 using a blowing nozzle B1 in the presence of an auxiliary air stream;

(2) performing a spunbond process involving heating and melting a thermoplastic resin to enter a spinning device; in the spinning device, dispersing the high-temperature thermoplastic resin into melt streams to be extruded from spinneret plates C1, C1′ and cooled by side blowing of cold air D1, D1′; stretching spunbond fibers using stretching devices E1, E1′; and joining the spunbond fibers with two side surfaces of the wood pulp fiber web 12, so as to form a multi-layer fiber web 15 having spunbond long fiber layers 13, 14 at its two sides and having the wood pulp fiber web 12 in its middle. The fiber webs in the multi-layer fiber web 15 are bound together using a pair of hot rolling rollers F1 that engage with each other, so as to form the spunbond wipe 10 having the upper and lower layers of the spunbond long fiber layers 13, 14 and having the intermediate layer of the wood pulp fiber web 12. A weight percentage of the wood pulp fiber web 12 in the spunbond wipe 16 is 75 wt %.

As shown in FIG. 2, the present invention further discloses a spunbond wipe made using the foregoing manufacturing method. The spunbond wipe has a laminated structure. The spunbond wipe 10 comprises upper and lower layers of spunbond long fiber layers 13, 14 and an intermediate layer of a wood pulp fiber web 12. Spunbond long fibers of the spunbond long fiber layers 13, 14 are woven into the wood pulp fiber web 12.

By adopting the aforementioned technical schemes, since the surface layers are the spunbond long fiber layers 13, 14 with longer and finer fibers, the fibers of the surface layers are less broken and arranged densely, so that the spunbond wipe is soft and unlikely to pill and fuzz. The spunbond long fibers are woven into the wood pulp fiber web 12, so that the wood pulp fibers adjacent to the surface layers are retained in the spunbond long fiber layers 13, 14. The spunbond long fiber layers 13, 14 on the surfaces are thinner and arranged densely, which can effectively prevent the wood pulp fibers from moving from the intermediate layer to the surfaces of the spunbond wipe and remaining on an object to be wiped.

### Embodiment 2

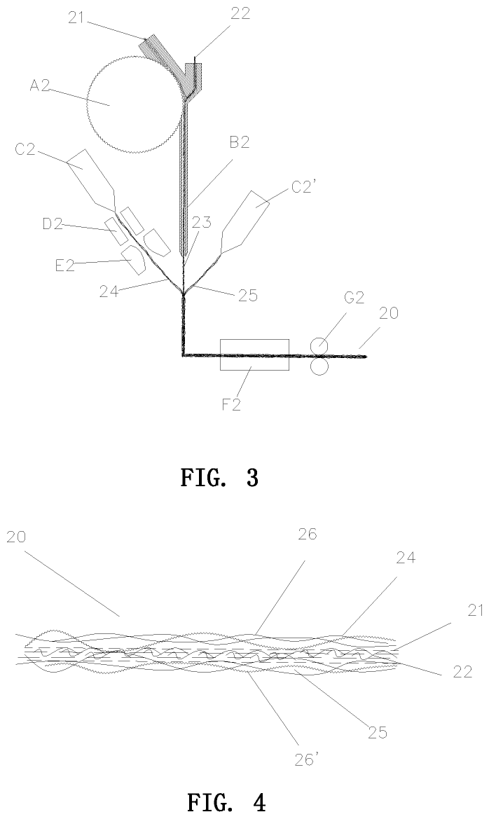

As shown in FIG. 3, the present invention discloses a manufacturing method for a spunbond wipe, comprising the following steps of:

(1) mixing wood pulp 21 that has been opened and scattered by an opening roller A2 with a hot-melt adhesive substance 22 into a mixture, and blowing the mixture using a blowing nozzle B2 in the presence of an auxiliary air stream to form a wood pulp fiber web 23 containing the hot-melt adhesive substance 22;

(2) performing a spunbond process for one side of the wood pulp fiber web 23, involving heating and melting two thermoplastic resins having a melting-point difference therebetween of ≥20° C. to enter a spinning device; in the spinning device, dispersing the high-temperature thermoplastic resins into melt streams to be extruded from a spinneret plate C2 and cooled by side blowing of cold air D2, thereby forming a spunbond long fiber layer 24 containing spunbond fibers each having a low-melting-point resin at its surface using a stretching device E2, wherein the spunbond fibers each having the low-melting-point resin at its surface are one-component spunbond fibers, two-component spunbond fibers, or a blend of the two; performing a melt blowing process for the other side of the wood pulp fiber web 23, involving heating and melting two thermoplastic resins having a melting-point difference therebetween of ≥20° C.; dispersing melt streams of the thermoplastic resins that have been extruded from a spinneret plate C2′ into fiber bundles having a diameter of ≤10 μm using a high-temperature, high-speed hot air stream, thereby forming a melt-blown fiber layer 25 in the presence of the hot air stream, wherein, the melt-blown fiber layer 25 contains melt-blown fibers each having a low-melting-point resin at its surface, the melt-blown fibers each having the low-melting-point resin at its surface may be one-component melt-blown fibers, two-component melt-blown fibers, or a blend of the two;

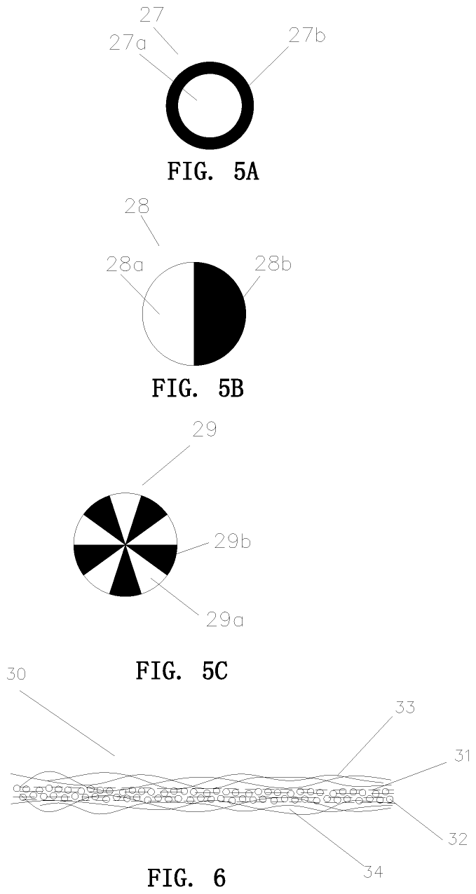

(3) performing a stretching process and joining the spunbond long fiber layer and the melt-blown fiber layer with two side surfaces of the wood pulp fiber web 23 containing the hot-melt adhesive substance 22, so as to form a multi-layer fiber web having the spunbond long fiber layer 24 at its one side and the melt-blown fiber layer 25 at its the other side and having the wood pulp fiber web 23 in its middle. Wherein, as shown in FIG. 5A through FIG. 5C, the two-component spunbond long fibers or two-component melt-blown fibers may be skin-core spunbond or melt-blown fibers 27, two-component segmented-pie spunbond or melt-blown fibers 28, or two-component side-by-side spunbond or melt-blown fibers 29. Besides, for the skin-core spunbond or melt-blown fiber 27, its skin layer resin 27b has a melting point less than 140° C. and lower than a melting point of its core layer resin 27a. For the two-component segmented-pie spunbond or melt-blown fiber 28 and two-component side-by-side spunbond or melt-blown fiber 29, one of their constituent resin 28b, 29b has a melting point less than 140° C. and lower than a melting point of the other constituent resin 28a, 29a. The spinneret plates C2, C2′ have two-component spinneret orifices. The two-component spinneret orifices of the spinneret plates C2, C2′ are arranged in a skin-core form, a segmented-pie form, or a side-by-side form; and

(4) binding the fiber webs in the multi-layer fiber web together using a hot air oven F2 and a pair of hot rolling rollers G2 that engage with each other, so as to form a spunbond wipe 20 having the upper and lower layers, one of which is the spunbond long fiber layer 24 and the other is the melt-blown fiber layer 25, and having the intermediate layer of the wood pulp fiber web 23 containing the hot-melt adhesive substance 22, wherein the spunbond long fiber layer 24 and the melt-blown fiber layer 25 contain spunbond long fibers 26 each having a low-melting-point resin at its surface and melt-blown fibers 26′ each having a low-melting-point resin at its surface, respectively. A weight percentage of the wood pulp fiber web 23 in the spunbond wipe 20 is 80 wt %. The hot-melt adhesive substance 22 may be polyester fibers having a low-melting-point, ES short fibers, two-component spunbond long fibers or a blend thereof. The blend refers to a blend of two or all of polyester fibers having a low-melting-point, ES short fibers and two-component spunbond long fibers.

As shown in FIG. 4, the present invention further discloses a spunbond wipe made using the foregoing manufacturing method. The spunbond wipe has a laminated structure. The spunbond wipe 20 comprises an upper layer of a spunbond long fiber layer 24, a lower layer of a melt-blown fiber layer 25, and an intermediate layer of a wood pulp fiber web 23. Spunbond long fibers of the spunbond long fiber layer 24 and melt-blown fibers of the melt-blown fiber layer 25 are woven into the wood pulp fiber web 23. The melt-blown fiber layer 25 contains one-component melt-blown fibers each having a high-melting-point resin at its surface, fibers each having a low-melting-point resin at its surface, or blend fibers of the two. The spunbond long fiber layer 24 contains one-component spunbond fibers each having a high-melting-point resin at its surface, spunbond fibers each having a low-melting-point resin at its surface, or blend fibers of the two. The high-melting-point resin and the low-melting-point resin have a melting-point difference therebetween of ≥20° C. The spunbond fibers each having the low-melting-point resin at its surface are one-component spunbond fibers, two-component spunbond fibers, or a blend of the two. The two-component spunbond fibers may be two-component skin-core spunbond fibers 27, two-component segmented-pie spunbond fibers 28, or two-component side-by-side spunbond fibers 29. A weight percentage of the wood pulp fiber web 23 in the spunbond wipe 20 is greater than 80 wt %.

By adopting the aforementioned technical schemes, since the spunbond long fiber layer 24 or the melt-blown fiber layer 25 contain the spunbond long fibers 26 each having a low-melting-point resin at its surface or the melt-blown fibers 26′ each having a low-melting-point resin at its surface, when the low-melting-point resin in the spunbond long fibers and the melt-blown fibers is melted in the hot air oven F2, the fibers are bound together. This not only enhances the overall strength of the resulting wipe, but also increases the wear-resistance of the wipe, so that the resulting wipe is more wear resistant and unlikely to pill and fuzz during the wiping process. Besides, the intermediate layer of the wood pulp fiber web 23 contains the hot-melt adhesive substance 22. The surface of the hot-melt adhesive substance 22 will melt and bind together when passing through the hot air oven F2, thereby effectively binding the wood pulp fibers and preventing the wood pulp fibers from moving to the outer layers of the wipe.

### Embodiment 3

As shown in FIG. 6, the present invention discloses a spunbond wipe 30. The spunbond wipe has a laminated structure. The spunbond wipe 30 comprises upper and lower layers of spunbond long fiber layers 33, 34 and an intermediate layer of a wood pulp fiber web 31. Spunbond long fibers of the spunbond long fiber layers 33, 34 are woven into the wood pulp fiber web 31. A super absorbent resin 32 is added to the intermediate layer of the wood pulp fiber web 31. Because the wood pulp fiber web 31 of the intermediate layer of the spunbond wipe 30 contains the super absorbent resin 32, the super absorbent resin 32 can quickly absorb water or detergent when the wipe is wetted with water or detergent for use, as it prevents the water or the detergent from being excessively transferred to the object to be wiped, which could otherwise degrade the wiping effect. During wiping, by virtue of the liquid retention ability of the super absorbent resin 32, the water or the detergent is slowly released to the surface of an object to be cleaned, such that stains can be rapidly removed due to the wetness of the wipe. Besides, the surface layers are the spunbond long fiber layers 33, 34 with longer and finer fibers, the fibers of the surface layers are less broken and arranged densely, so that the spunbond wipe is soft and unlikely to pill and fuzz.

### Embodiment 4

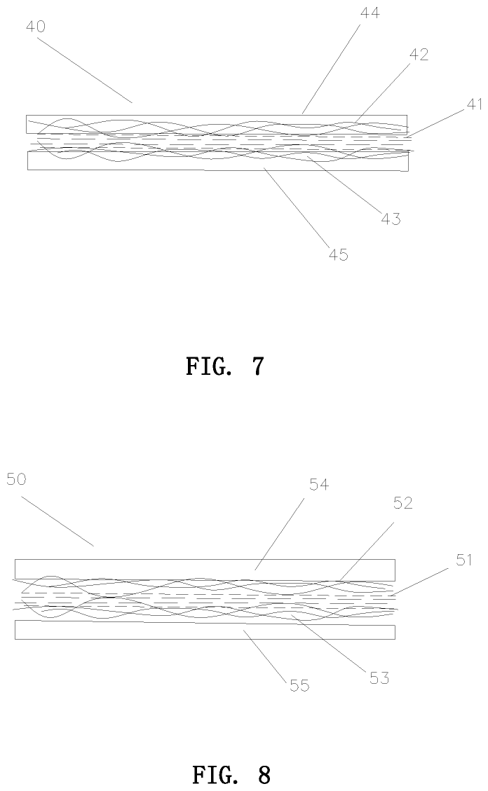

As shown in FIG. 7, the present invention discloses a spunbond wipe 40. The spunbond wipe has a laminated structure. The spunbond wipe 40 comprises upper and lower layers of spunbond long fiber layers 42, 43 and an intermediate layer of a wood pulp fiber web 41. Spunbond long fibers of the spunbond long fiber layers 42, 43 are woven into the wood pulp fiber web 41. An adhesive 44, 45 is attached to the surfaces of the spunbond long fiber layers 42, 43 and to the interiors of the spunbond long fiber layers 42, 43 that are adjacent to the surfaces.

After the fiber webs are laid and before the webs are solidified, an adhesive spraying process is performed using blowing nozzles in the presence of a high-pressure hot air flow to spray the adhesive 44, 45 on two side surfaces of the multi-layer fiber web having the spunbond long fiber layers at its two sides and having the wood pulp fiber web in its middle. As a result, the adhesive 44, 45 is attached to the surfaces of the spunbond long fiber layers 42, 43. Since the adhesive spraying process is performed before the spunbond long fiber layers 42, 43 are not bound, during the adhesive spraying process, the adhesive will not only adhere to the surfaces of the spunbond long fiber layers 42, 43 but also penetrate into the interiors of the spunbond long fiber layers 42, 43 that are adjacent to the surfaces, so that the adhesive is attached to the surfaces of the spunbond long fiber layers 42, 43 and to the interiors of the spunbond long fiber layers 42, 43 that are adjacent to the surfaces. An adhesive film is formed on each of the surfaces of the spunbond long fiber layers 42, 43, such that when in use, fuzz and pilling of the spunbond long fibers on the surfaces caused by friction are prevented, and the wood pulp short fibers in the intermediate layer is also further prevented from falling out to cause “linting” during use.

### Embodiment 5

As shown in FIG. 8, the present invention discloses a spunbond wipe 50. The spunbond wipe has a laminated structure. The spunbond wipe 50 comprises upper and lower layers of spunbond long fiber layers 52, 53 and an intermediate layer of a wood pulp fiber web 51. Spunbond long fibers of the spunbond long fiber layers 52, 53 are woven into the wood pulp fiber web 51.

An adhesive 54, 55 is attached to the surfaces of the spunbond long fiber layers 52, 53.

After the fiber webs are solidified, a roll coating process is performed to coat the adhesive 54, 55 on two side surfaces of the multi-layer fiber web having the spunbond long fiber layers 52, 53 at its two sides and having the wood pulp fiber web 51 in its middle, so that the adhesive 54, 55 is attached to the surfaces of the spunbond long fibers. Since the adhesive 54, 55 is attached to the surfaces of the spunbond long fiber layers 52, 53, an adhesive film is formed on each of the surfaces of the spunbond long fiber layers 52, 53, such that when in use, fuzz and pilling of the spunbond long fibers on the surfaces caused by friction are prevented, and the wood pulp short fibers in the intermediate layer is also further prevented from falling out to cause “linting” during use.

What is claimed is:

1. A spunbond wipe, comprising upper and lower layers and an intermediate layer of a wood pulp fiber web, at least one of the upper and lower layers being a spunbond long fiber layer, wherein spunbond long fibers of the spunbond long fiber layer are woven into the wood pulp fiber web.

2. The spunbond wipe of claim 1, wherein one of the upper and lower layers is the spunbond long fiber layer, and the other is a melt-blown fiber layer.

3. The spunbond wipe of claim 1 or 2, wherein the spunbond long fiber layer contains one-component spunbond fibers each having a high-melting-point resin at its surface, spunbond fibers each having a low-melting-point resin at its surface, or blend fibers of the two.

4. The spunbond wipe of claim 3, wherein the high-melting-point resin and the low-melting-point resin have a melting-point difference therebetween of ≥20° C.

5. The spunbond wipe of claim 3, wherein the spunbond fibers each having the low-melting-point resin at its surface are one-component spunbond fibers, two-component spunbond fibers, or a blend of the two.

6. The spunbond wipe of claim 5, wherein the two-component spunbond fibers are two-component skin-core spunbond fibers, two-component segmented-pie spunbond fibers, or two-component side-by-side spunbond fibers.

7. The spunbond wipe of claim 2, wherein the melt-blown fiber layer contains one-component melt-blown fibers each having a high-melting-point resin at its surface, fibers each having a low-melting-point resin at its surface, or blend fibers of the two.

8. The spunbond wipe of claim 7, wherein the fibers each having the low-melting-point resin at its surface are one-component melt-blown fibers, two-component melt-blown fibers, or a blend of the two.

9. The spunbond wipe of claim 8, wherein the two-component melt-blown fibers are two-component skin-core melt-blown fibers, two-component segmented-pie melt-blown fibers, or two-component side-by-side melt-blown fibers.

10. The spunbond wipe of claim 1, wherein a weight percentage of the wood pulp fiber web in the spunbond wipe is greater than 50 wt %.

11. The spunbond wipe of claim 1, wherein a weight percentage of the wood pulp fiber web in the spunbond wipe is 65 wt %-80 wt %.

12. The spunbond wipe of claim 1, wherein the wood pulp fiber web contains a hot-melt adhesive substance.

13. The spunbond wipe of claim 12, wherein the hot-melt adhesive substance is polyester fibers having a low-melting-point, ES short fibers, two-component spunbond long fibers or a blend thereof.

14. The spunbond wipe of claim 1, wherein the wood pulp fiber web contains a super absorbent resin.

15. The spunbond wipe of claim 1, wherein an adhesive is attached to surfaces of the upper and lower layers of the spunbond wipe.

16. The spunbond wipe of claim 1, wherein an adhesive is attached to surfaces of the upper and lower layers of the spunbond wipe and to interiors of the upper and lower layers that are adjacent to the surfaces.

17. The spunbond wipe of claim 14 or 15, wherein the adhesive is hot-melt adhesive or solvent adhesive.

18. A manufacturing method for the spunbond wipe of claim 1, comprising steps of:
(1) opening and scattering wood pulp using an opening roller, and forming a wood pulp fiber web using a blowing nozzle in the presence of an auxiliary air stream;
(2) performing a spunbond process for one side of the wood pulp fiber web, involving heating and melting a thermoplastic resin to enter a spinning device; in the spinning device, dispersing the high-temperature thermoplastic resin into melt streams to be extruded from spinneret plates and cooled by side blowing of cold air; stretching spunbond fibers using stretching devices; and joining the spunbond fibers with two side surfaces of the wood pulp fiber web, so as to form a multi-layer fiber web having the spunbond long fiber layer at its at least one side and having the wood pulp fiber web in its middle; and
(3) binding the fiber webs in the multi-layer fiber web together using a heating device, so as to form the spunbond wipe having one least one of the upper and lower layers of the spunbond long fiber layer and having the intermediate layer of the wood pulp fiber web.

19. The manufacturing method of claim 18, in the step (2), performing the spunbond process for one side of the wood pulp fiber web, and performing a melt blowing process for another side of the wood pulp fiber web.

20. The manufacturing method of claim 19, in the step (2), performing the spunbond process for one side of the wood pulp fiber web, involving heating and melting two thermoplastic resins having a melting-point difference therebetween of ≥20° C. to enter the spinning device; in the spinning device, dispersing the high-temperature thermoplastic resins into melt streams to be extruded from the spinneret plates; cooling by side blowing of the cold air to form the spunbond long fiber layer using stretching devices, wherein the spunbond long fiber layer contains fibers each having a high-melting-point resin at its surface, fibers each having a low-melting-point resin at its surface, or blend fibers of the two.

21. The manufacturing method of claim 20, wherein the spinneret plates have two-component spinneret orifices.

22. The manufacturing method of claim 21, wherein the two-component spinneret orifices of the spinneret plates are arranged in a skin-core form, a segmented-pie form, or a side-by-side form.

23. The manufacturing method of claim 19, in the step (2), performing the melt blowing process for the other side of the wood pulp fiber web, involving heating and melting two thermoplastic resins having a melting-point difference therebetween of ≥20° C.; dispersing melt streams of the thermoplastic resins that have been extruded from a spinneret plate into fiber bundles having a diameter of ≤10 μm using a high-temperature, high-speed hot air stream, thereby forming a melt-blown fiber layer in the presence of the hot air stream, wherein the melt-blown fiber layer contains fibers each having a high-melting-point resin at its surface, fibers each having a low-melting-point resin at its surface, or blend fibers of the two.

24. The manufacturing method of claim 18, in the step (1), mixing the wood pulp that has been opened and scattered by the opening roller with a hot-melt adhesive substance into a mixture, and blowing the mixture using the blowing nozzle in the presence of the auxiliary air stream to form the wood pulp fiber web containing the hot-melt adhesive substance.

25. The manufacturing method of claim 18, before the step (3), performing an adhesive spraying process using blowing nozzles in the presence of a high-pressure hot air flow to spray an adhesive on two side surfaces of the multi-layer fiber web.

26. The manufacturing method of claim 18, after the step (3), performing a roll coating process to coat an adhesive on two side surfaces of the multi-layer fiber web.

27. The manufacturing method of claim 18, wherein in the step (3) the heating device is a hot air oven, a hot rolling roller or a combination thereof.

28. The manufacturing method of claim 18, in the step (1), mixing the wood pulp that has been opened and scattered by the opening roller with a super absorbent resin into a mixture, and blowing the mixture using the blowing nozzle in the presence of the auxiliary air stream to form the wood pulp fiber web containing the super absorbent resin.
